(19) United States
(12) Patent Application Publication
(10) Pub. No.: US 2026/0158200 A1
(43) Pub. Date: Jun. 11, 2026

(54) STENT-GRAFT BASED PULSATILE BLOOD FLOW CATHETER

(71) Applicant: Oceansio Holdings LLC, Las Vegas, NV (US)

(72) Inventors: Yijiang Li, Dublin, CA (US); Summer Xiatian Li, La Canada Flintridge, CA (US)

(21) Appl. No.: 19/375,429

(22) Filed: Oct. 31, 2025

Related U.S. Application Data
(60) Provisional application No. 63/714,854, filed on Oct. 31, 2024.

(51) Int. Cl. A61M 1/36; A61M 39/24; A61M 39/00

(52) U.S. Cl. CPC A61M 1/3659; A61M 39/24; A61M 2039/0009; A61M 2202/0021; A61M 2202/0413; A61M 2205/3331; A61M 2210/127

(57) ABSTRACT

Provided is a stent-graft based pulsatile blood flow catheter. Through dual unidirectional blood flow direction control members and an expandable stent-graft design of the catheter, sufficient heart support can be provided, while a trauma to blood can be greatly reduced. In addition, the catheter has a simple structure and can be implanted through a minimally invasive surgery, so that a severe trauma caused by an extracorporeal blood circulation system to a patient is avoided. Furthermore, the catheter can entirely or partially replace the heart’s pumping function and minimize blood damage, thereby providing a more effective and safer treatment option for patients suffering from cardiogenic shock or cardiac failure.

### CROSS REFERENCE TO RELATED APPLICATIONS

The present application claims the benefit of U.S. Provisional Patent Application No. 63/714,854 filed on October 31, 2024, the contents of which are incorporated herein by reference in their entirety.

### TECHNICAL FIELD

The present application belongs to the technical field of medical devices, and particularly relates to a stent-graft based pulsatile blood flow catheter.

### BACKGROUND OF THE INVENTION

Existing devices for treating cardiogenic shock and heart failure can be classified into two categories: in-vivo blood circulation devices and extracorporeal blood circulation devices. The extracorporeal blood circulation devices, such as ECMO, draw blood out of the body and pump the blood through an external large-scale machine. Although this type of devices can effectively maintain blood circulation, they are difficult to use for a long term due to their large size and significant blood damage. Meanwhile, the extracorporeal blood circulation devices require external interfaces, which can cause significant trauma to patients and are inconvenient to carry.

The in-vivo blood circulation devices, such as intra-aortic balloon counter pulsation devices and left ventricular axial flow pumps, are placed in the body to support pumping of a heart by providing additional power to the blood. Although these devices are smaller and may cause less trauma to the patient, their support to the heart is limited due to constraints in design, and thus they can only partially replace the function of the heart and are inadequate for providing sufficient support in cases of severe heart failure. The in-vivo devices may also cause significant blood damage.

### SUMMARY

To solve or relieve the above technical problems, the present application provides a brand-new design which is a stent-graft based pulsatile blood flow catheter. The catheter is a heart supporting device which can completely support the heart or partially support the heart when needed, causes small damage to blood, and is easy to implant inside the body.

In a first aspect, the embodiments of the present application provide a stent-graft based pulsatile blood flow catheter. The catheter includes a first catheter unit and a second catheter unit which are connected to each other.

A diameter of the second catheter unit is smaller than a diameter of the first catheter unit; a first blood flow direction control member is arranged in the first catheter unit; the first blood flow direction control member is located at a position of the first catheter unit away from the second catheter unit; and the first blood flow direction control member is configured such that blood can flow into the first catheter unit through the first blood flow direction control member, but is prevented from flowing out of the first catheter unit through the first blood flow direction control member

A blood outflow channel is on a side wall of the first catheter unit; a second blood flow direction control member is located at a position of the first catheter unit corresponding to the blood outflow channel; wherein the second blood flow direction control member is configured blood can only flow out of the blood outflow channel through the second blood flow direction control member.

In a preferred embodiment of the present application, a third catheter unit is located at one end of the first catheter unit away from the second catheter unit, and a diameter of the third catheter unit is smaller than the diameter of the first catheter unit.

In a preferred embodiment of the present application, the blood outflow channel includes a flow guide structure and a blood outlet.

The flow guide structure is located at an outer side of the first catheter unit, and the flow guide structure is connected to the side wall of the first catheter unit; the blood outlet is located at one end of the flow guide structure away from a joint of the flow guide structure with the first catheter unit; the second blood flow direction control member is located at the joint of the flow guide structure and the first catheter unit, or at one end of the flow guide structure closer to the first catheter unit.

In a preferred embodiment of the present application, the flow guide structure is a tubular structure.

In a preferred embodiment of the present application, a cross section of the first blood flow direction control member is the same as a cross section of the first catheter unit, and a cross section of the second blood flow direction control member is the same as a cross section of the flow guide structure.

In a preferred embodiment of the present application, the blood outflow channel is located at two respective and opposite positions of two sides of the first catheter unit.

In a preferred embodiment of the present application, the flow guide structure and the second blood flow direction control member are both located in the first catheter unit; the flow guide structure is connected to the side wall of the first catheter unit; the flow guide structure is located at one side of the second blood flow direction control member adjacent to the first blood flow direction control member; and the blood outlet is located at the side wall of the first catheter unit.

When the second blood flow direction control member is open, blood flows out of the catheter through the second blood flow direction control member, the flow guide structure, and the blood outlet in sequence.

When the second blood flow direction control member is closed, the second blood flow direction control member is connected to the flow guide structure, and the blood is prevented from flowing out of the catheter.

In a preferred embodiment of the present application, the flow guide structure and the blood outlet are both arranged on the side wall of the first catheter unit; the second blood flow direction control member is arranged on the outer side of the first catheter unit and correspond to the blood outlet; and the second blood flow direction control member is connected to the side wall of the first catheter unit.

When the second blood flow direction control member is open, blood flows out of the catheter through the blood outlet, the flow guide structure, and the second blood flow direction control member in sequence.

When the second blood flow direction control member is closed, blood is prevented from flowing out of the catheter.

In a preferred embodiment of the present application, the first blood flow direction control member and the second blood flow direction control member are both valves.

In a preferred embodiment of the present application, the first catheter unit, the second catheter unit, and the third catheter unit are formed by stent-grafts.

In a preferred embodiment of the present application, a sheath sleeves one end of the second catheter unit away from the first catheter unit; and the sheath is insertable into a living blood vessel for connection to an extracorporeal blood circulation system.

The present application provides a stent-graft based pulsatile blood flow catheter. Through dual unidirectional blood flow direction control members and an expandable stent-graft design of the catheter, sufficient heart support can be provided, while a trauma to blood can be greatly reduced. In addition, the catheter has a simple structure and can be greatly reduced. In addition, the catheter has a simple structure and can be implanted through a minimally invasive surgery, so that a severe trauma caused by an extracorporeal circulation device to a patient is avoided. Furthermore, the catheter can entirely replace the heart's pumping function and minimize blood damage, thereby providing a more effective and safer treatment options for patients suffering from cardiogenic shock or cardiac failure.

In a second aspect, the embodiments of the present application further provide a method of using a pulsatile blood flow catheter to assist in cardiac pumping function. The method is implemented by the pulsatile blood flow catheter described in the first aspect. The method includes:

placing the first catheter unit into an ascending aorta to cause blood that flows from a left ventricle into the ascending aorta to at least partially flow into the first catheter unit through the first blood flow direction control member, and then to flow into the extracorporeal blood circulation system through the second catheter unit; and

Processing, via the extracorporeal blood circulation system, the blood that flows into the extracorporeal blood circulation system such that the processed blood flows out of the extracorporeal blood circulation system, through the second catheter unit, the first catheter unit, and the blood outflow channel in sequence.

Compared with the prior art, the method of using a pulsatile blood flow catheter to assist in the heart’s pumping function provided by the present application has the same beneficial effects as the stent-graft based pulsatile blood flow catheter provided in the first aspect, and will not be repeated here.

### BRIEF DESCRIPTION OF THE DRAWINGS

The accompanying drawings described herein are provided to further illustrate the present application and form a part of the present application. The illustrative embodiments and their explanations of the present application are used to explain the present application and do not constitute an improper limitation on the present application. The following text will describe in detail some specific embodiments of the present application in an exemplary manner rather than limitation with reference to the accompanying drawings. The same reference numerals in the accompanying drawings indicate the same or similar components or parts. Those skilled in the art should understand that these accompanying drawings are not necessarily drawn to scale. In the accompanying drawings:

FIG. 1 is a schematic diagram of a catheter according to Embodiment I of the present application.

FIG. 2 is a schematic diagram of a catheter according to embodiment II of the present application.

FIG. 3 is a schematic diagram of a catheter according to Embodiment III of the present application.

FIG. 4 is a schematic diagram of a catheter according to Embodiment IV of the present application.

FIG. 5 is a schematic diagram of a catheter according to Embodiment V of the present application.

FIG. 6 is a schematic diagram of a catheter according to Embodiment IV of the present application.

FIG. 7 and FIG. 8 are diagrams showing the usage state of some embodiments of the present application.

### DETAILED DESCRIPTION OF EMBODIMENTS

In order to help those skilled in the art better understand the solutions of the present application, the technical solutions in the embodiments of the present application are clearly and completely described below with reference to the accompanying drawings in the embodiments of the present application. Apparently, the described embodiments are merely some embodiments of the present application, rather than all of them. Any other embodiments obtained by those skill in the art based on the embodiments of the present application without making creative efforts shall fall within the protection scope of the present application.

The applicant has found through research that although existing in-vivo circulation devices are placed in a body, their support to the heart is insufficient, and can only partially replace the function of the heart, making it difficult to provide a sufficient support in severe cardiac failure situations. In addition, for the sake of miniaturization design, the in-vivo circulation devices often cause significant blood damage. Moreover, there is currently no heart supporting device that can fully support a heart, cause minimal blood damage, and be easily implanted in a body.

To solve the above problems, it is necessary to provide a device that can be implanted into a body and cause minimal damage to blood.

Therefore, a catheter structure of the present application is provided.

The following provides a detailed introduction to the catheter structure provided by the present application:

### Embodiment I

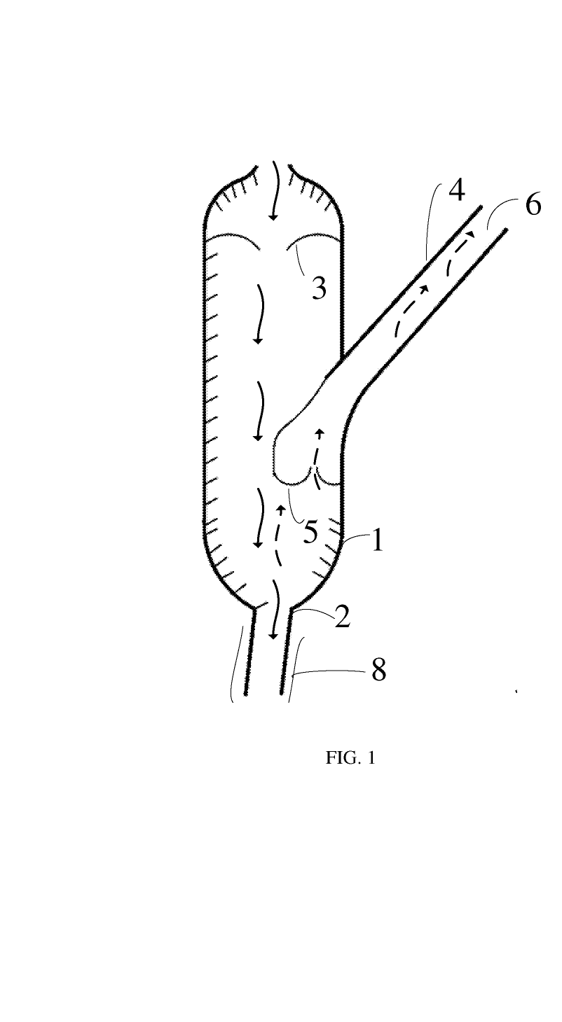

As shown in FIG. 1, A catheter of this embodiment includes a first catheter unit 1 and a second catheter unit 2 which are connected to each other. Namely, the catheter of the present application includes two catheter units connected to each other, and the two catheter units form an in-vivo blood circulation device connectable to an extracorporeal blood circulation system to provide life support for a patient.

A diameter of the second catheter unit 2 is smaller than a diameter of the first catheter unit 1. A first blood flow direction control member 3 is located in the first catheter unit 1. The first blood flow direction control member 3 is located at a position of the first catheter unit 1 away from the second catheter unit 2. The first blood flow direction control member 3 is configured such that blood can flow into the first catheter unit 1 through the first blood flow direction control member 3 but is prevented from flowing out of the first catheter unit 1 through the first blood flow direction control member 3. Specifically, the first blood flow direction control member 3, the second blood flow direction control member 5, and the first catheter unit 1 jointly define a space for temporarily storing blood, namely, an amount of blood each time the catheter pumps in or out. Therefore, preferably, the first blood flow direction control member 3 should be located at one end of the first catheter unit 1 adjacent to the heart to maximize this space for temporarily storing blood.

In some embodiments of the present application, the diameter of the first catheter unit 1 can be set according to a deficiency of the patient’s heart pumping ability, to compensate for the insufficient pumping ability and ensure the normal pumping function. If there is a severe deficiency/decline in the pumping ability of the heart, the diameter of the first catheter unit 1 can be larger, without considering blockage of an original pumping channel of the heart.

In some embodiments of the present application, one end of the first catheter unit 1 away from the second catheter unit is placed in an ascending aorta. To allow blood entering the body from the extracorporeal blood circulation system to flow out of the catheter and to respective organs of the living body, a blood outflow channel is located at a side wall of the first catheter unit 1; a second blood flow direction control member 5 is located at a position of the first catheter unit 1 corresponding to the blood outflow channel; and the second blood flow direction control member 5 is configured such that blood can only flow out of the blood outflow channel through the second blood flow direction control member 5. A control direction of the first blood flow direction control member 3 is different from a control direction of the second blood flow direction control member 5.

The blood outflow channel includes a flow guide structure 4 and a blood outlet 6.

The flow guide structure 4 is located at an outer side of the first catheter unit 1, and the flow guide structure 4 is connected to the side wall of the first catheter unit 1. The blood outlet 6 is located at one end of the flow guide structure 4 away from a joint of the flow guide structure 4 with the first catheter unit 1. The second blood flow direction control member 5 is located at the joint between the flow guide structure 4 and the first catheter unit 1, or at one end of the flow guide structure 4 adjacent to the first catheter unit 1. Specifically, first, the second blood flow direction control member 5 can be physically oriented towards the heart, away from the heart, or even at a 90-degree angle relative to a blood flow direction of a large blood vessel. In a specific application, a direction and a position of the blood outlet 6 are further affected by physiological factors. Since large blood vessels have many branch vessels that deliver blood to important organs connected to the large blood vessel, during a heart failure, in order to ensure the delivery of the blood to these organs, it is desired to set the flow guide structure 4 to be oriented towards these branch vessels. In this way, blood can be guided better in the respective flow directions of these branch vessels and ensure the delivery of the blood to the organs. These branch vessels include: coronary arteries for supplying blood to the heart, branch vessels on an aortic arch for supplying blood to the brain, and superior mesenteric arteries, artery trunks, and renal arteries which supply blood to the stomach and intestines and the kidneys respectively. Specifically, the orientation of the flow guide structure 4 and the orientation of the blood outlet 6 do not affect blood flow, and the blood outlet 6 can be positioned near a portion, which needs blood, in a human blood vessel.

In this embodiment, a central axis of the first blood flow direction control member 3 and a central axis of the second blood flow direction control member 5 are both parallel to a central axis of the first catheter unit 1.

Specifically, the flow guide structure 4 is a tubular structure.

In some embodiments of the present application, to adapt to the diameter of the first catheter unit 1, a cross section of the first blood flow direction control member 3 is the same as a cross section of the first catheter unit 1. Meanwhile, to adapt to a diameter of the flow guide structure 4, a cross section of each second blood flow direction control member 5 is the same as a cross section of the flow guide structure 4.

In this embodiment, the cross section of the flow guide structure 4 is smaller than the cross section of the first catheter unit. Therefore, the cross section of the second blood flow direction control member 5 is smaller than the cross section of the first blood flow direction control member 3. Specifically, a diameter of the cross section of the second catheter unit 2 is about 1 cm, and a diameter of the cross section of the first catheter unit 1 is 2.5 to 3.5 cm.

In some embodiments of the present application, to make the blood in the first catheter unit 1 flow out more smoothly, two blood outflow channels can be located at two opposite positions at two sides of the first catheter unit 1 respectively.

In some embodiments of the present application, the first blood flow direction control member 3 and the second blood flow direction control member 5 are both valves. The valves are artificial membrane-like structures that can be open and closed in the first catheter unit 1, and have biocompatibility. The valves function as one-way valves, so that the blood can flow only in a direction and is prevented from flowing in a reverse direction. The valves and the catheter are disposable consumables. In another embodiment, the first blood flow direction control member 3 and the second blood flow direction control members 5 can also be one-way valves, baffles, or one-way rolling ball valves. Any structures that allow blood to flow in one direction and restricts blood flow in the reverse direction fall within the protection scope of the present application. Specifically, the valve material can be decellularized porcine or bovine pericardial tissues, or a high polymer material.

In some embodiments of the present application, to connect the catheter to an extracorporeal blood circulation system, a sheath 8 sleeves one end of the second catheter unit 2 away from the first catheter unit 1. The sheath 8 is located in a living blood vessel for connection to the extracorporeal blood circulation system.

### Embodiment II

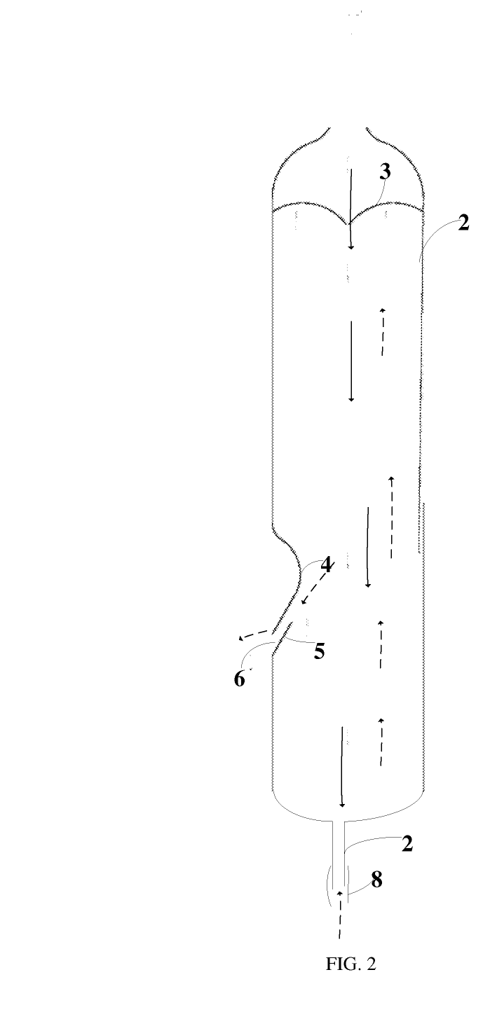

As shown in FIG. 2, the difference between Embodiment II and Embodiment I is that the flow guide structure 4 is located at an inner side of the first catheter unit 1; the blood outlet 6 is located at the side wall of the first catheter unit 1; the second blood flow direction control member 5 is located in the first catheter unit 1; and the flow guide structure 4 is located at one side of the second blood flow direction control members 5 adjacent to the first blood flow direction control member 3. In this embodiment, the central axis of the second blood flow direction control member 5 forms a 90-degree angle with the central axis of the first blood flow direction control member 3. When the second blood flow direction control member 5 is open, blood flows out of the catheter through the second blood flow direction control members 5, the flow guide structure 4, and the blood outlet 6 in sequence.

When the second blood flow direction control members 5 is closed, the second blood flow direction control member 5 is in contact with the flow guide structure 4, and blood is prevented from flowing out of the catheter.

Furthermore, the flow guide structure 4 and the blood outlet 6 are both located at the side walls of the first catheter unit 1. The second blood flow direction control member 5 is arranged at the outer side of the first catheter unit 1 and corresponds to the blood outlet 6.

When the second blood flow direction control member 5 is open, blood flows out of the catheter through the second blood flow direction control member 5, the blood outlet 6, and the flow guide structure 4 in sequence.

When the second blood flow direction control member 5 is closed, blood is prevented from flowing out of the catheter.

### Embodiment III

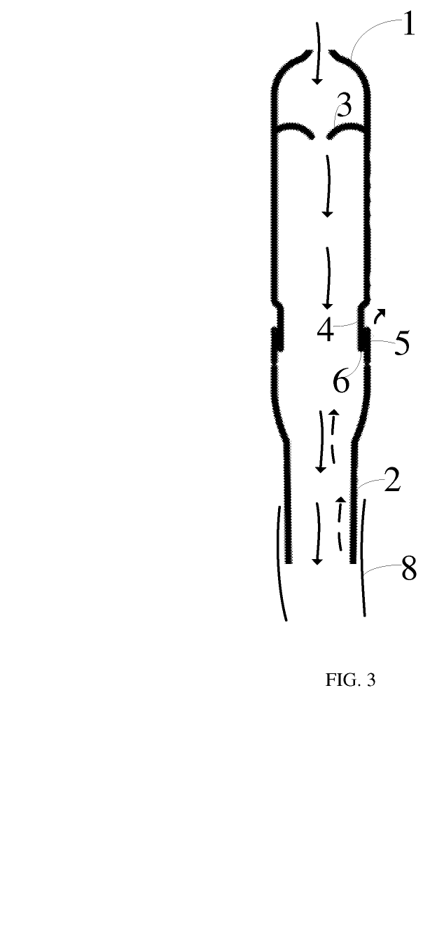

As shown in FIG. 3, the difference of Embodiment III from Embodiment I and Embodiment II is that the flow guide structure 4 and the blood outlet 6 are both arranged at the side wall of the first catheter unit 1. The second blood flow direction control member 5 is located at the outer side of the first catheter unit 1, corresponding to the blood outlet 6.

When the second blood flow direction control member 5 is open, blood flows out of the catheter through the blood outlet 6, the flow guide structure 4, and the second blood flow direction control member 5 in sequence.

When the second blood flow direction control members 5 is closed, blood is prevented from flowing out of the catheter.

Embodiment II is taken as an example to explain the working principle of the catheters of Embodiment I, Embodiment II, and Embodiment III in detail. When the extracorporeal blood circulation system starts to work, the catheter draws blood from the left ventricle 9 by a power system of the extracorporeal blood circulation system, and opens the first blood flow direction control member 3 by utilizing power of the blood flow. When the first blood flow direction control member 3 is open, blood entering the first catheter unit 1 enters the second catheter unit 2 through the first blood flow direction control member 3, and then enters the extracorporeal blood circulation system through the second catheter unit 2. After being processed by the extracorporeal blood circulation system, the blood is drawn by the power system of the extracorporeal blood circulation system, and the second blood flow direction control member 5 is open by the blood flow. Next, the blood flows out of the catheter through the blood outflow channel and is supplied to different organs of the living body, thus completing blood circulation. The extracorporeal blood circulation system specifically includes a blood pump, an oxygenator, and an extracorporeal blood control main unit. The extracorporeal blood control main unit controls the blood pump to draw blood out of the body, and the blood is delivered into the body after oxygenation in the oxygenator.

### Embodiment IV

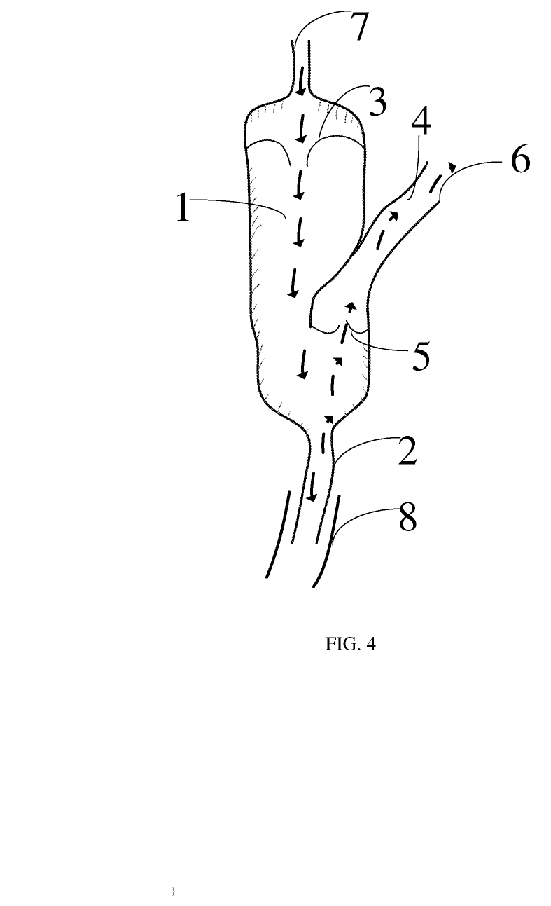

As shown in FIG. 4, further to Embodiment I, a third catheter unit 7 is arranged at one end of the first catheter unit 1 away from the second catheter unit 2, and a diameter of the third catheter unit 7 is smaller than the diameter of the first catheter unit 1. Specifically, the third catheter unit 7 is formed by a stent-graft too. The first catheter unit 1 may be located at a distance of 4 cm, 8 cm, 10 cm, 20 cm, 30 cm, or even 40 cm from the distal end (one end away from the heart) of the third catheter unit. In addition, there may be more than one first catheter unit.

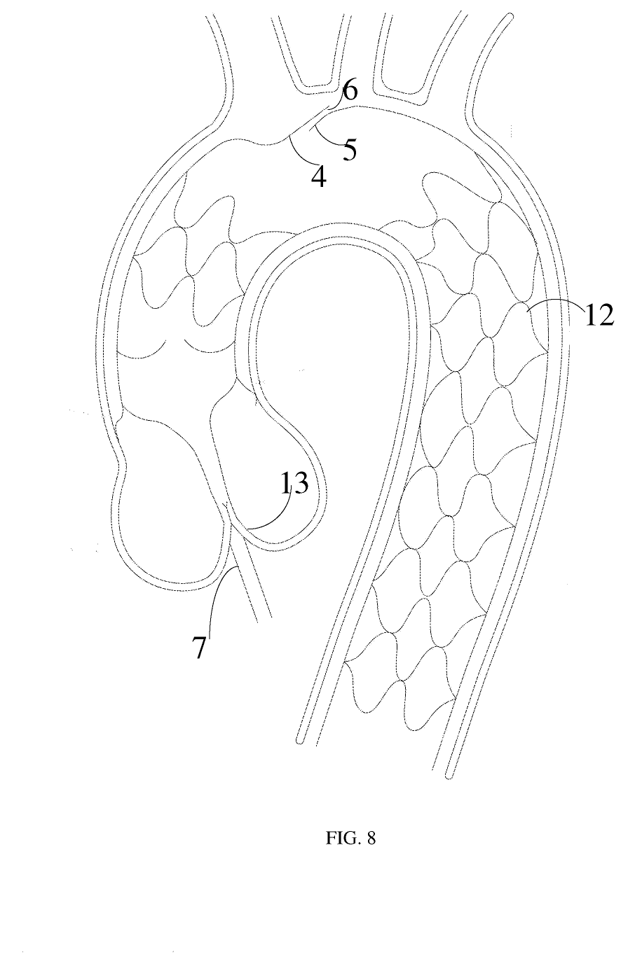

As shown in FIG. 8, it is necessary to ensure that after the third catheter unit 7 is placed in an aortic valve 13, the aortic valve 13 can still open and close properly, so that the third catheter unit 7 needs to have a smaller diameter. In addition, since a sheath 8 sleeves one end of the second catheter unit 2 away from the first catheter unit 1, the sheath 8 is located in a living blood vessel for connection to an extracorporeal blood circulation system, and the blood vessel of the patient is relatively thin, the second catheter unit 2 needs to have a smaller diameter to match the diameter of the living blood vessel. Therefore, in the present application, the diameter of the second catheter unit 2 is smaller than the diameter of the first catheter unit 1 too. Specifically, the diameter of the cross section of the second catheter unit 2 is between 1.5 cm and 0.7 cm.

### Embodiment V

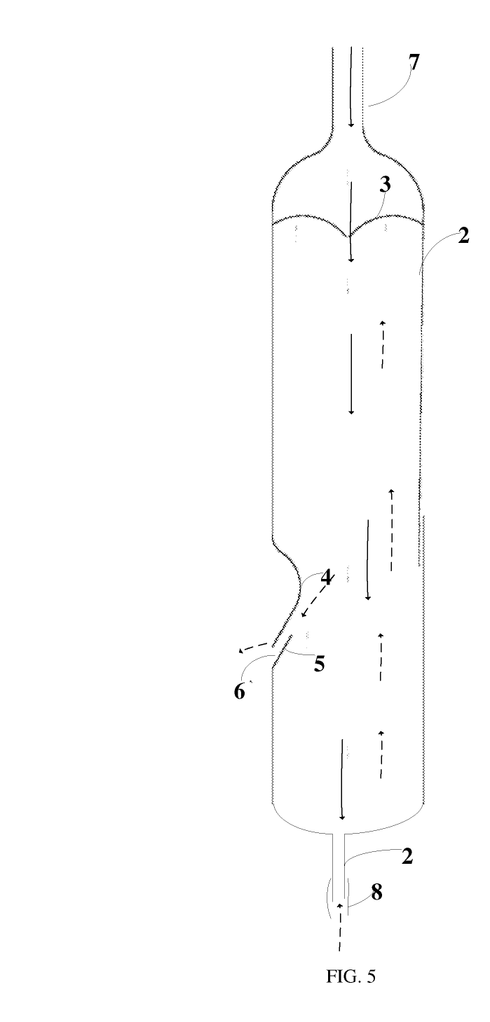

As shown in FIG. 5, Further to Embodiment II, a third catheter unit 7 is located at one end of the first catheter unit 1 away from the second catheter unit 2, and the diameter of the third catheter unit 7 is smaller than the diameter of the first catheter unit 1. The catheter of the present application is implanted into a human body, with one end of the catheter entering the left ventricle 9 through an aortic valve to draw blood from the left ventricle 9. The entire catheter is fixed in an ascending aorta.

### Embodiment VI

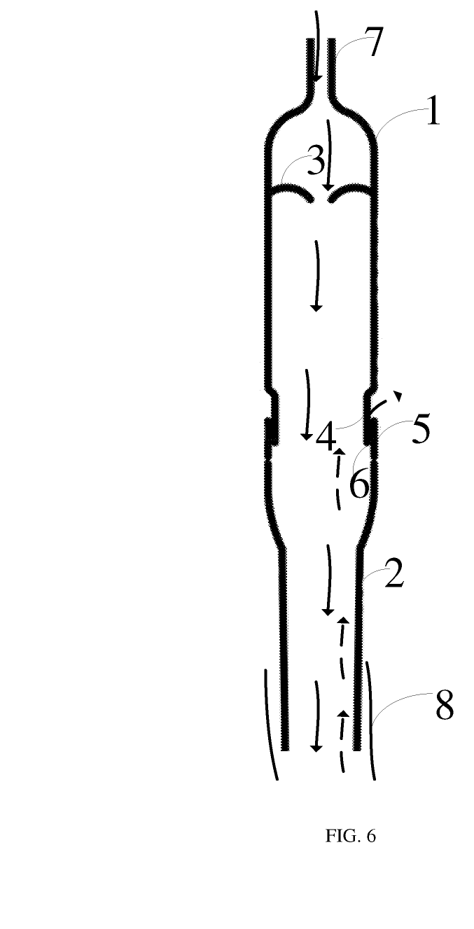

As shown in FIG. 6, Further to Embodiment III, a third catheter unit 7 is located at one end of the first catheter unit 1 away from the second catheter unit 2, and the diameter of the third catheter unit 7 is smaller than the diameter of the first catheter unit 1. The catheter of the present application is implanted into a human body, and one end of the catheter enters the left ventricle 9 through an aortic valve to draw the blood out from the left ventricle 9. The entire catheter is fixed in an ascending aorta.

The position of the first catheter unit 1 in Embodiment IV, Embodiment V, and Embodiment VI is fixed depending firstly on the requirement that the catheter of the present application should remain connected to a blood vessel connected to the blood pump of the extracorporeal blood circulation system, so that the position of the catheter of the present application is maintained through this connection relationship. In addition, the rigidity of the catheter can also help maintain the position of the first catheter unit 1.

After fixing the first catheter unit 1, it is also necessary to maintain the normal opening and closing of the aortic valve of the patient. An implementation method is as follows. The diameter of the third catheter unit 7 is set to be small enough to avoid affecting the normal opening and closing of the aortic valve.

### Embodiment VII

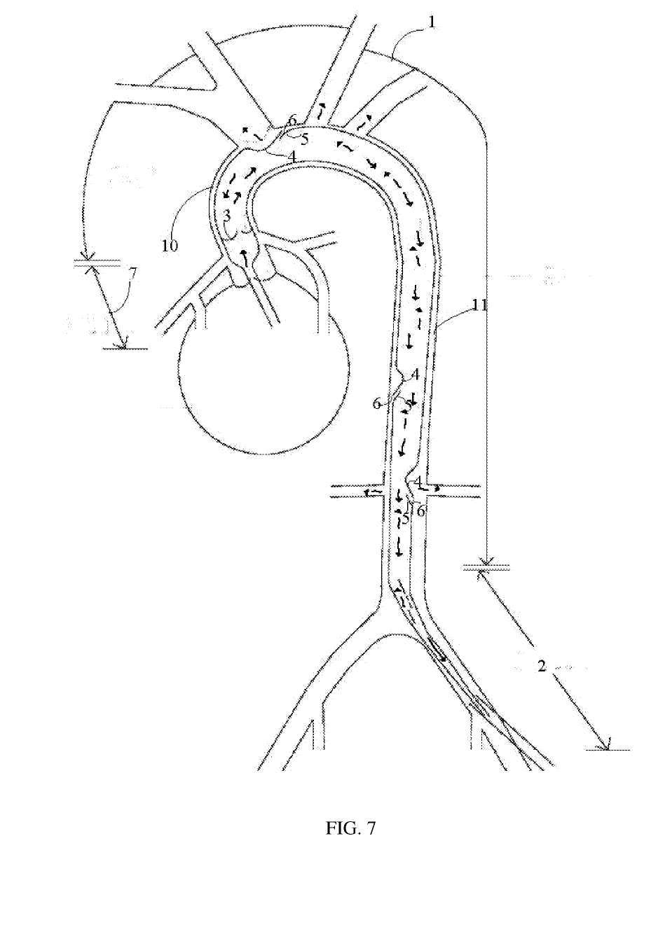

As shown in FIG. 7, the working principle of the catheters provided in Embodiment IV, Embodiment V, and Embodiment VI will be explained in detail below. The entire catheter is fixed in an ascending aorta 10. Then, the catheter is connected to the extracorporeal blood circulation system after passing through a descending aorta 11. When the extracorporeal blood circulation system starts to work, the catheter draws blood from the left ventricle 9 by the power system of the extracorporeal blood circulation system, and opens the first blood flow direction control member 3 by the blood flow. When the first blood flow direction control member 3 is open, the blood entering the third catheter unit 7 enters the first catheter unit 1 through the first blood flow direction control member 3, and then enters the extracorporeal blood circulation system through the second catheter unit 2. After the extracorporeal blood circulation system processes the blood, the processed blood is delivered back into the body by the power system of the extracorporeal blood circulation system, and the second blood flow direction control member 5 is open by the blood flow. Next, the blood flows out of the catheter through the blood outflow channel and is supplied to different organs of the living body, thus completing blood circulation.

As shown in FIG. 8, the catheter of the present application is implanted into the human body, with one end of the catheter entering the left ventricle 9 through the aortic valve to draw blood out of the left ventricle 9. The entire catheter is fixed in the ascending aorta 10. The first catheter unit 1, the second catheter unit 2, and the third catheter unit 7 are all formed by a stent-graft 12 in some embodiments of the present application. The stent-graft 12 can be placed into the blood vessel in a folded state, and then automatically expand. The stent-graft 12 is usually used in vascular intervention, structural heart diseases, urology, respiration, and other fields. A coating material needs to have good biocompatibility and various desired physical properties, chemical properties, and other properties. Through an expandable structure of the catheter, the device can adapt to diameters of different blood vessels, ensuring the stability of the device in the human body and causing no excessive pressure on the blood vessels. The covering material can be electrospun (similar to a mask) or woven, such as a polytetrafluoroethylene (PTFE), core tex, or coated darcon polyester woven material. A stent material is nickel titanium alloy. The stent-graft is made of an alloy material with limited expansion, expanded via balloon dilation or self-expansion, to ensure that the stent-graft may not excessively expand and apply excessive pressure to the blood vessel.

To adapt to the diameters of different blood vessels, without causing an excessive pressure on the blood vessels, a series of types of catheters can be manufactured based on an understanding and benchmarking of sizes and diameters of physiological blood vessels of an adult. In addition, the stent-graft can be kept slightly smaller than the physiological blood vessels in size to ensure that the stent-graft may not adhere to the vascular walls and may not apply pressure to the blood vessels. According to the physiological characteristic of human physiological blood vessels, which gradually narrow from one end adjacent to the heart towards one end away from the heart, the stent-graft of the catheter tapers gradually or stepwise from one end closer to the heart to one end away from the heart, ensuing that it does not apply an excessive pressure to the blood vessel.

In design, the embodiments of the present application overcome the problems that the existing in-vivo blood circulating devices cannot completely support heart and causes significant blood damage. Through dual unidirectional valve structures and an expandable stent-graft design of the catheter of the present application, the catheter can provide sufficient support to heart and greatly reduce blood damage. Since no high-speed rotating component such as an impeller is needed in the present application, and the opening-and-closing frequency of the first blood flow direction control member 3 and the second blood flow direction control member 5 is close to the pulsating rhythm of human heart, thus avoiding any structures that may cause blood damage. In addition, the first blood flow direction control member 3 and the second blood flow direction control member 5 are made of high-biocompatibility materials such as tissue valves, bovine pericardial valves, porcine valves, high-molecular polymer valves, and membrane-covered fabric valves. Extensive data and user experience have shown that the use of these widely used high-biocompatible materials can cause less blood damage. In summary, the catheter of the present application can significantly reduce damage to blood components.

The catheter of the present application is simple and can be implanted through a minimally invasive surgery, thereby avoiding serious trauma to the patient caused by the extracorporeal circulation devices. Moreover, the catheter of the present application can be placed in the body of the patient, which not only entirely replaces the pumping function of the heart, but also minimizes blood damage, thereby providing a more effective and safer treatment option for patients suffering from cardiogenic shock and cardiac failure.

It should be finally noted that the foregoing various embodiments are merely intended to describe the technical solutions of the present application, and should not be construed as limiting. Although the present application is described in detail with reference to the foregoing various embodiments, those skilled in the art should understand that they may still make modifications to the technical solutions described in the foregoing embodiments or make equivalent replacements to partial or all technical features thereof. However, these modifications or replacements do not make the essence of the corresponding technical solutions depart from the scope of the technical solutions of the various embodiments of the present application.

What is claimed is:

1. A stent-graft based pulsatile blood flow catheter, comprising:
a first catheter unit and a second catheter unit connected to the first catheter, wherein a diameter of the second catheter unit is smaller than a diameter of the first catheter unit, the first catheter unit comprises a first blood flow direction control member, the first blood flow direction control member is located at a position in the first catheter unit away from the second catheter unit; the first blood flow direction control member is configured such that blood can flow into the first catheter unit through the first blood flow direction control member, but is prevented from flowing out of the first catheter unit through the first blood flow direction control member;
a blood outflow channel on a side wall of the first catheter unit;
a second blood flow direction control member located at a position of the first catheter unit corresponding to the blood outflow channel, wherein the second blood flow direction control member is configured such that blood can only flow out of the blood outflow channel through the second blood flow direction control member.

2. The stent-graft based pulsatile blood flow catheter according to claim 1, wherein a third catheter unit is located at one end of the first catheter unit away from the second catheter unit, and a diameter of the third catheter unit is smaller than the diameter of the first catheter unit.

3. The stent-graft based pulsatile blood flow catheter according to claim 1, wherein the blood outflow channel comprises a flow guide structure and a blood outlet;
wherein the flow guide structure is located at an outer side of the first catheter unit, and the flow guide structure is connected to the side wall of the first catheter unit; the blood outlet is located at one end of the flow guide structure away from a joint of the flow guide structure with the first catheter unit; the second blood flow direction control member is located at the joint of the flow guide structure and the first catheter unit, or at one end of the flow guide structure closer to the first catheter unit.

4. The stent-graft based pulsatile blood flow catheter according to claim 3, wherein the flow guide structure is a tubular structure.

5. The stent-graft based pulsatile blood flow catheter according to claim 3, wherein a cross section of the first blood flow direction control member is the same as a cross section of the first catheter unit, and a cross section of the second blood flow direction control member is the same as a cross section of the flow guide structure.

6. The stent-graft based pulsatile blood flow catheter according to claim 1, wherein the blood outflow channel is located at two respective and opposite positions of two sides of the first catheter unit.

7. The stent-graft based pulsatile blood flow catheter according to claim 3, wherein the flow guide structure and the second blood flow direction control member are both located in the first catheter unit; the flow guide structure is connected to the side wall of the first catheter unit; the flow guide structure is located at one side of the second blood flow direction control member adjacent to the first blood flow direction control member; and the blood outlet is located at the side wall of the first catheter unit;
when the second blood flow direction control member is open, blood flows out of the catheter through the second blood flow direction control member, the flow guide structure, and the blood outlet in sequence; and
when the second blood flow direction control member is closed, the second blood flow direction control member is connected to the flow guide structure, and blood is prevented from flowing out of the catheter.

8. The stent-graft based pulsatile blood flow catheter according to claim 3, wherein the flow guide structure and the blood outlet are both arranged on the side wall of the first catheter unit, the second blood flow direction control member is arranged on the outer side of the first catheter unit and correspond to the blood outlet; the second blood flow direction control member is connected to the side wall of the first catheter unit;
when the second blood flow direction control members is open, blood flows out of the catheter through the blood outlet, the flow guide structure, and the second blood flow direction control member in sequence; and
when the second blood flow direction control member is closed, blood is prevented from flowing out of the catheter.

9. The stent-graft based pulsatile blood flow catheter according to claim 1, wherein the first blood flow direction control member and the second blood flow direction control member are both valves.

10. The stent-graft based pulsatile blood flow catheter according to claim 2, wherein the first catheter unit, the second catheter unit, and the third catheter unit are all formed by stent-grafts.

11. The stent-graft based pulsatile blood flow catheter according to claim 2, wherein a sheath sleeves one end of the second catheter unit away from the first catheter unit; and the sheath is insertable into a living blood vessel for connection to an extracorporeal blood circulation system.

12. A method of using a pulsatile blood flow catheter to assist in cardiac pumping, wherein the method is implemented by the pulsatile blood flow catheter according to claim 1, and wherein the method comprises:
placing the first catheter unit into an ascending aorta to cause blood that flows from a left ventricle into the ascending aorta to at least partially flow into the first catheter unit through the first blood flow direction control member, and then to flow into the extracorporeal blood circulation system through the second catheter unit; and
processing, via the extracorporeal blood circulation system, the blood that flows into the extracorporeal blood circulation system such that the processed blood flows out of the extracorporeal blood circulation system through the second catheter unit, the first catheter unit, and the blood outflow channel in sequence.
